(19) United States
(12) Patent Application Publication
(10) Pub. No.: US 2015/0353481 A1
(43) Pub. Date: Dec. 10, 2015

(54) OXIDATIVE CARBONYLATION OF PRIMARY AMINES TO ISOCYCANATES CATALYZED BY GROUP X DIVALENT NOBLE METAL COMPOUNDS OR COMPLEXES

(71) Applicants: Jean-Jacques Katz, Las Vegas, NV (US); Anny Jutand, Paris (FR)

(72) Inventors: Jean-Jacques Katz, Las Vegas, NV (US); Anny Jutand, Paris (FR)

(21) Appl. No.: 14/732,047

(22) Filed: Jun. 5, 2015

Related U.S. Application Data
(60) Provisional application No. 62/008,230, filed on Jun. 5, 2014.

(51) Int. Cl. C07C 263/00

(52) U.S. Cl. CPC C07C 263/00; C07C 2101/14

(57) ABSTRACT

Processes for the preparation of monoisocyanates, diisocyanates or polyisocyanates of the present invention are described which comprises reacting monomeric or oligomeric primary amines or diamines, and carbon monoxide at atmospheric pressure in the presence of a catalytic amount of a compound or a complex of a divalent metal selected from palladium, nickel and platinum, and in the presence of stoichiometric or catalytic quantities of benzoquinone as an oxidant. Isocyanates are produced in high yields and purities. They can be isolated as free isocyanates or as blocked isocyanates by in situ reaction with blocking agents that contain an active hydrogen being attached to oxygen, sulfur or nitrogen. Monoisocyanates and diisocyanates serve as chemical, agricultural or pharmaceutical intermediates. Diisocyanates and polyisocyanates are used to produce polyurethanes, polyureas, polyisocyanurates and related polymers.

### REFERENCE TO RELATED APPLICATION

This application claims priority to U.S. Provisional Patent Application Ser. No. 62/008,230, filed Jun. 5, 2014, the entire content of which is incorporated herein by reference.

### FIELD OF THE INVENTION

The present invention pertains to the production of organic isocyanates.

### BACKGROUND OF THE INVENTION

Organic isocyanates are major industrial chemicals for the polyurethane industry. Hereinafter, and for the purpose of this invention, the word isocyanate is meant to include monoisocyanates, diisocyanates, and polyisocyanates, unless otherwise stated. The two largest isocyanates, by volume, are aryl diisocyanates: TDI (2,4 and 2,6-toluene diisocyanate isomers), monomeric or pure MDI (4,4′-, 2,4′- and 2,2′-methylene diphenyl diisocyanate isomers), and polymeric MDI (PMDI). These are commercially converted into polyurethanes, polyureas, polyurethanes/ureas, polyisocyanaurates and other derived polymers.

TDI is mostly used in the manufacture of flexible polyurethane foams, primarily utilized as cushioning material in furniture and transportation applications.

PMDI is mostly used to manufacture rigid polyurethane foams that are widely utilized as thermal insulating materials in construction, consumer appliances, industrial, packaging and other applications. Rigid foam is PMDI's largest application segment.

MDI, as pure MDI or 4,4′ MDI, is used in non-foam applications such as elastomers, paints & coatings, adhesives, encapsulants and sealants.

Aliphatic diisocyanates, such as H12 MDI (4,4′-methylene dicyclohexyl diisocyanate), HMDI (hexamethylene diisocyanate), CHDI (1,4-cyclohexyldiisocyanate) and IPDI (isophorone diisocyanate) are produced in much smaller volumes than TDI or MDI, but are widely used in paint, coating and sealing applications where their durability, abrasion resistance and light stability are important benefits in the construction and outdoor sport surfaces industries.

Aryl and aliphatic monoisocyanates, for their part, are important building blocks and intermediates in the biochemical, pharmaceutical, agricultural and specialty chemical industries.

Commercial isocyanate production technology today is exclusively based on the reaction of phosgene (COCl₂) with an organic primary amine or diamine (phosgenation), to yield an intermediate carbamoyl chloride. This moiety is subsequently thermally decomposed to the corresponding isocyanate. Hydrogen chloride, a highly corrosive and toxic gas of very little commercial value, is generated as a byproduct.

In this expensive and dangerous process, phosgene is used as a feedstock component. Phosgene is a very toxic gas that can result in major safety hazards. It is also a controversial material because it can be, and has been, used as a chemical warfare agent. Consequently, production processes relying on phosgene are banned from many regions.

There is a continuing need for new processes which convert primary amines directly to isocyanates.

### SUMMARY OF THE INVENTION

Methods for production of an isocyanate are provided according to aspects of the present invention which include reacting a homogeneous reaction mixture including a monomeric or oligomeric primary amine or diamine, carbon monoxide, a Group X divalent noble metal compound or complex catalyst, a quinone oxidizing agent and a polar aprotic solvent, thereby producing an isocyanate. According to aspects, the reaction mixture is substantially phosgene free. According to further aspects, the carbon monoxide is at atmospheric pressure.

Methods for production of an isocyanate are provided according to aspects of the present invention which include reacting a homogeneous reaction mixture comprising a monomeric or oligomeric primary amine or diamine, carbon monoxide, a Group X divalent noble metal compound or complex catalyst, an oxidizing agent and a polar aprotic solvent, thereby producing an isocyanate, wherein the reaction mixture is substantially CuCl₂ free and substantially HCl free. According to aspects, the reaction mixture is substantially phosgene free. According to further aspects, the carbon monoxide is at atmospheric pressure.

According to aspects of the invention, the Group X divalent noble metal compound or complex catalyst includes nickel(II), platinum(II) and/or palladium(II).

Aspects of methods for production of an isocyanate of the present invention are described which further include reacting the isocyanate with a blocking agent, producing a blocked isocyanate.

A catalyst is present in the reaction mixture in a catalytic amount. According to aspects of methods for production of an isocyanate of the present invention, a catalyst is present in an amount in the range of 0.05% to 2% per mole of the monomeric or oligomeric primary amine or diamine.

According to aspects of methods for production of an isocyanate of the present invention, a catalyst is present in an amount in the range of 0.1% to 1% per mole of the monomeric or oligomeric primary amine or diamine.

According to aspects of methods for production of an isocyanate of the present invention, the reacting is under reaction conditions including a temperature in the range of 0° C.-150° C., such as a temperature in the range of 25° C.-120° C.

According to aspects of methods for production of an isocyanate of the present invention, the oxidizing agent is a quinone.

According to aspects of methods for production of an isocyanate of the present invention, the quinone oxidizing agent is present in a stoichiometric amount per mole of the monomeric or oligomeric primary amine or diamine.

Optionally, the quinone oxidizing agent is present in an amount in the range of 0.05% to 2% per mole of the monomeric or oligomeric primary amine or diamine, and a stoichiometric amount of manganese oxide is present relative to the amount of the monomeric or oligomeric primary amine or diamine.

### DETAILED DESCRIPTION OF THE INVENTION

The singular terms “a,” “an,” and “the” are not intended to be limiting and include plural referents unless explicitly stated otherwise or the context clearly indicates otherwise.

Methods according to aspects of the present invention are one-step methods for direct production of isocyanates from primary amines or diamines in a homogeneous oxidative carbonylation process, without requiring phosgene, in the presence of a Group X divalent noble metal compound or complex catalyst, a quinone oxidizing agent and a polar aprotic solvent.

Methods according to aspects of the present invention include reacting a reaction mixture which includes: a monomeric or oligomeric primary amine or diamine; carbon monoxide; a Group X divalent noble metal compound or complex catalyst; a quinone oxidizing agent; and a polar aprotic solvent, under reaction conditions, thereby producing an isocyanate.

Methods according to aspects of the present invention include reacting a reaction mixture which includes: a monomeric or oligomeric primary amine or diamine; carbon monoxide; a Group X divalent noble metal compound or complex catalyst; a quinone oxidizing agent; and a polar aprotic solvent, under reaction conditions, thereby producing an isocyanate, wherein the reaction mixture is substantially phosgene free.

The term “substantially phosgene free” refers to a reaction mixture containing less than 10%, less than 5%, less than 1% or less than 0.1% phosgene in a method according to aspects of the present invention.

Methods according to aspects of the present invention include reacting a reaction mixture which includes: a monomeric or oligomeric primary amine or diamine; carbon monoxide; a Group X divalent noble metal compound or complex catalyst; a quinone oxidizing agent; and a polar aprotic solvent, under reaction conditions, thereby producing an isocyanate. wherein the reaction mixture is substantially free of CuCl₂ and substantially free of HCl.

According to particular aspects of the present invention, the Group X divalent noble metal compound or complex catalyst is a nickel(II), platinum(II) or palladium(II) compound or complex catalyst.

According to preferred aspects of the present invention, the reaction is run in the presence of carbon monoxide at atmospheric pressure and in the absence of added oxygen.

According to preferred aspects of the present invention, the reaction is run in the presence of carbon monoxide at atmospheric pressure, in the absence of added oxygen and in the absence of phosgene.

An oxidizing agent included in a reaction mixture according to aspects of the present invention is a quinone. The quinone is included in stoichiometric quantities and is subsequently reduced to hydroquinone, as an oxidizing agent for the catalytic carbonylation reaction. Use of a quinone as an oxidizing agent according to aspects of methods of the present invention and not oxygen eliminates stoichiometric amounts of water formed when oxygen is used as an oxidizing agent, which promotes urea formation to the detriment of isocyanates.

Representative quinones that may be used as oxidizing agents in this invention include, but are not limited to, 1,4-benzoquinone, 1,4-naphtoquinone, 9,10-anthraquinone, 1,2-benzoquinone, and 1,2-naphtaquinone. Quinones substituted at positions adjacent to the ketone groups with electron-withdrawing groups, which include, but are not limited to CN, SO₃, Cl and Br, are further examples of suitable oxidants for this invention. Fully substituted quinones, such as, but not limited to, 2,3-Dichloro-5,6-dicyano-1,4-benzoquinone (DDQ), 2,3,5,6-tetrachloro-1,4-benzoquinone (p-chloranil), 3,4,5,6-tetrachloro-1,2-benzoquinone (o-chloranil), and 3,4,5,6-tetrabromo-1,2-benzoquinone (o-bromanil) are further examples of strong oxidants that may be used in the invention.

More preferred quinones are 1,2-benzoquinone and 1,4-benzoquinone, with 1,4-benzoquinone being the most preferred oxidant in the invention, due to low cost, low toxicity, ease of handling and commercial availability.

An included quinone oxidizing agent removes 2 electrons from Pd metal, re-oxidizing it to Pd(II), and also captures/removes the 2 protons of the amine to produce the hydroquinone, thereby yielding the isocyanate without any by-product, such as hydrogen chloride which would need to be neutralized. Thus, methods according to aspects of the present invention include reacting a reaction mixture which includes: a monomeric or oligomeric primary amine or diamine; carbon monoxide; a Group X divalent noble metal compound or complex catalyst; an oxidizing agent; and a polar aprotic solvent, under reaction conditions, thereby producing an isocyanate, wherein the reaction mixture is substantially free of CuCl₂ and substantially free of HCl.

A further preferred method of this invention involves the use of said quinone oxidizing agent in catalytic amounts in the presence of stoichiometric amounts of manganese oxide (MnO₂) relative to the amounts of monomeric or oligomeric primary amine or diamine. Manganese oxide (Mn (IV)) oxidizes hydroquinone, by capturing/removing 2 protons, back to the starting quinone and is converted to manganese hydroxide Mn(OH)₂ (Mn(II)), which precipitates out of solution, an added benefit of this method.

The term “substantially free of CuCl₂” refers to a reaction mixture containing less than 10%, less than 5%, less than 1% or less than 0.1% CuCl₂ in a method according to aspects of the present invention.

The term “substantially free of HCl” refers to a reaction mixture containing less than 10%, less than 5%, less than 1% or less than 0.1% HCl in a method according to aspects of the present invention.

The term “catalytic amount” is well-known and refers to an amount that is less than a stoichiometric amount of a product-limiting reactant included in the catalyzed reaction. According to aspects of the present invention, a catalytic amount is from 0.00001 mol % to 90 mol % of the moles of the starting monomeric or oligomeric primary amine or diamine in the reaction mixture, such as 0.0001 mol % to 50 mol %, 0.001 mol % to 10 mol %, 0.05 mol % to 2 mol % or 0.1 mol % to 1 mol % of the starting monomeric or oligomeric primary amine or diamine in the reaction mixture.

The term “blocking group” as used herein refers to a temporary substituent used to reversibly modify and thereby protect a reactive functional group from chemical reaction.

Reaction conditions are those conditions under which chemical oxidative carbonylation reaction of primary amines to isocyanates catalyzed by group X divalent noble metal compounds or complexes occurs, and includes, for example, conditions of temperature, pressure and presence and amount of catalyst and reactant.

Methods of oxidative carbonylation reaction of primary amines to isocyanates catalyzed by group X divalent noble metal compounds or complexes according to aspects of the present invention is carried out under atmospheric pressure, although more or less pressure may be used, such as 0.1-100 atm.

Methods of oxidative carbonylation reaction of primary amines to isocyanates catalyzed by group X divalent noble metal compounds or complexes according to aspects of the present invention is carried out under inert atmosphere such as under a blanket of inert gas, exemplified by nitrogen, although other inert materials may be used.

In this specification, and for the purpose of this invention, the term primary amine includes primary aliphatic amines, primary aryl amines, primary alkaryl amines, primary aralkyl amines, primary aliphatic diamines, primary aryl diamines, primary alkaryl diamines and primary aralkyl diamines. The primary aryl, alkaryl and aralkyl diamines may be monomeric, oligomeric or polymeric.

Examples of amine starting materials include, but are not limited to, primary amines with the formula R—NH₂ where R is an organo group selected from the following: C₁₋₂₀ aliphatic, C₄₋₂₀ cycloaliphatic, C₆₋₂₀ aryl, C₇₋₃₀ aralkyl and C₇₋₃₀ alkaryl. More preferably, R is substituted, or preferably unsubstituted C₁₋₂₀ alkyl, C₂₋₂₀ alkenyl, C₂₋₂₀ alkynyl, C₄₋₂₀ cycloalkyl, C₄₋₂₀ cycloalkenyl, C₆₋₃₀ aryl, C₇₋₃₀ aralkyl and C₇₋₃₀ alkaryl.

Examples of diamine starting materials include, but are not limited to, primary diamines with the formula H₂N—R′—NH₂, where R′ is an organo group selected from the following: C₂₋₂₀ aliphatic, C₄₋₂₀ cycloaliphatic, C₆₋₂₀ aryl, C₇₋₃₀ aralkyl and C₇₋₃₀ alkaryl. More preferably, R′ is substituted, or preferably unsubstituted C₂₋₂₀ alkyl, C₃₋₂₀ alkenyl, C₃₋₂₀ alkynyl, C₄₋₂₀ cycloalkyl, C₄₋₂₀ cycloalkenyl, C₆₋₃₀ aryl, C₇₋₃₀ aralkyl and C₇₋₃₀ alkaryl.

Preferred aryl amines include, but are not limited to, aniline, p-toluidine, 2-, 3-, 4-methyl aniline, 1- and 2-naphtyl amine.

Preferred aryl diamines include, but are not limited to, 1,2-phenylene diamine, 1,3-phenylene diamine, 1,4-phenylene diamine; 2,4-toluene diamine, 2,6-toluene diamine and 3,5-toluene diamine; 1,5-naphtalene diamine, 2,6-naphtalene diamine; the various polycyclic diaminodiphenylmethane (DADPM) oligomers in the form of 3, 4 and 5 rings, and monomeric diaminodiphenylmethane, particularly 2,2′-diaminodiphenylmethane, 2,4′-diaminodiphenylmethane, and most particularly 4,4′-diaminodiphenylmethane (methylene dianiline or MDA).

Preferred aralkyl amines include, but are not limited to, benzylamine.

Preferred aliphatic diamines include, but are not limited to, 1,6-hexamethylene diamine, isophorone diamine, 1,4-cyclohexyldiamine and 4,4′-methylene dicyclohexyl diamine (H₁₂ MDA).

Solvent selection for the present invention is guided by the need for polarity and required reaction temperatures to achieve optimal reaction kinetics, isocyanate yield, purity, and product selectivity.

Most preferred solvents for optimal isocyanate yields and purities, are polar aprotic, linear or cyclic molecules. Such solvents include, but are not limited to, acetone, ethyl acetate, acetonitrile, N,N-dimethyl acetamide (DMAC), N,N-dimethyl formamide (DMF), dimethyl sulfoxide (DMSO), and N-methyl-2-pyrrolidone (NMP).

Other preferred solvents include, but are not limited to, chlorinated aryl compounds, including monochlorobenzene (MCB), 1,2-dichlorobenzene or ortho-dichlorobenzene (ODCB) and 1,3-dichlorobenzene or meta-dichlorobenzene (MDCB).

A Group X divalent noble metal catalyst, defined for the purpose of this invention either as catalyst precursor or catalyst precursor and ligand—having the following structure:

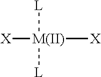

where M=Ni, Pd, Pt, X=C (Chloride), OAc (Acetate), Br (Bromide), OCOCF₃ (Trifluoroacetate), BF₄ (tetrafluoroborate) and L=Ligand as defined below (no Ligand=catalyst precursor) is used in methods according to aspects of the present invention, controlling the process at the isocyanate stage, while preventing urea production from the reaction of isocyanate with excess amine.

Selection of the catalyst allows for control of the process at the isocyanate stage, while preventing urea production from the reaction of isocyanate with excess amine.

Choice of ligand facilitates two steps of the catalytic cycle. The use of strong σ electron-donating ligands, such as trialkylphosphines, increases electron density around the metal, accelerating the oxidative addition of the catalyst to the substrate, believed to be the rate determining step. Choice of ligand also determines the mechanism by which oxidative addition occurs. The elimination step is accelerated by the use of sterically bulky ligands, in particular phosphine ligands exhibiting a large cone angle, known as Tolman angle. Phosphine ligands may be replaced with N-heterocyclic carbenes (NHCs).

On that basis, the catalytic species that may be used in this invention include, but are not limited to:

(1) a catalyst precursor including, but not limited to, Allylpalladium chloride dimer, Bis(acetonitrile)palladium(II) chloride, Bis(benzonitrile)palladium(II) chloride, Palladium(II) acetate, Palladium(II) bromide, Palladium(II) chloride, Palladium(II) trifluoroacetate, and Tetrakis(acetonitrile)palladium(II) tetrafluoroborate

(2) a complex formed in situ using a catalyst precursor, as described above, and the necessary organophosphine ligand including, but not limited to, Triphenylphosphine, Tri-(2-furyl)phosphine, Tri-o-tolylphosphine, Trimesitylphosphine, Tricyclohexylphosphine, Triisopropylphosphine, Tri-n-butylphosphine, Di-tert-butylmethylphosphine, Tri-tert-butylphosphine, 2-(Dicyclohexylphosphino)-2′-Isopropylbiphenyl, 2-(Dicyclohexylphosphino)-2′,4′,6′-Triisopropylbiphenyl, 2-(Di-tert-butylphosphino)biphenyl, 2-(Dicyclohexylphosphino)biphenyl, 2-Dicyclohexylphosphino-2′-(N,Ndimethylamino) biphenyl, 2-Diphenylphosphino-2′-(N,Ndimethylamino) Biphenyl, 2-(Dicyclohexylphosphino)-2′-methylbiphenyl, 2-(Di-tert-butylphosphino)-2′-methylbiphenyl, 2-Di-tert-butylphosphino-2′-(N,Ndimethylamino)biphenyl, 2-Dicyclohexylphosphino-2′,6′-diisopropoxy-1,1′-biphenyl, 2-Di-tert-butylphosphino-2′,4′,6′-Triisopropylbiphenyl, Bis(diphenylphosphino)methane, 1,2-Bis(diphenylphosphino)ethane, 1,2-Bis(dicyclohexylphosphino)ethane, 1,3-Bis(diphenylphosphino)propane, 1,3-Bis(dicyclohexylphosphino)propane, 1,4-Bis(diphenylphosphino)butane, 1,5-Bis(diphenylphosphino)pentane, Bis(2-diphenylphosphinophenyl)ether, 1,1′-Bis(diphenylphosphino)ferrocene, 1,1′-Bis(diisopropylphosphino)ferrocene, 1 1,1′-Bis(di-tert-butylphosphino)ferrocene, 1,2-Bis(diphenylphosphino)benzene, and 9,9-Dimethyl-4,5-bis(diphenylphosphino) xanthenes.

(3) a complex formed in situ using a catalyst precursor and N-heterocyclic carbenes (NHCs) ligands prepared from imidazolium and imidazolinium salts including, but not limited to, 1,3-Bis(2,4,6-trimethylphenyl)imidazolium chloride, 1,3-Bis(2,6-diisopropylphenyl)imidazolium chloride, 1,3-Bis(adamant-1-yl)imidazolium chloride, 1,3-Bis(2,4,6-trimethylphenyl)-4,5-dihydroimidazolium tetrafluoroborate, 1,3-Bis(2,6-diisopropylphenyl)imidazolidinium tetrafluoroborate, 1,3-Bis(2,4,6-trimethylphenyl)imidazolidinium chloride, and 1,3-Bis(2,6-diisopropylphenyl) imidazolidinium chloride, or

(4) introduced as a preformed catalyst including, but not limited to, [1,2-Bis(diphenylphosphino)ethane]palladium(II) chloride, Bis(triethylphosphine)palladium(II) chloride, Bis(tricyclohexylphosphine)palladium(II) chloride, Bis(methyldiphenylphosphine)palladium(II) chloride, Bis(methyldiphenylphosphine)palladium(II) chloride, [1,3-Bis(diphenylphosphino)propane]palladium(II) chloride, (1,5-cyclooctadiene)palladium(II) chloride, Bis(triphenylphosphine)palladium(II) acetate, Bis(triphenylphosphine)palladium(II) chloride, Bis[tri(o-tolyl)phosphine]palladium(II) chloride, [1,4-Bis(diphenylphosphino)butane]palladium(II) chloride and Bis(tricyclohexylphosphine) palladium(II) chloride.

Nickel (II) catalysts may also catalyze, under similar conditions, the oxidative carbonylation of primary amines reaction with the same efficiency as the more expensive palladium catalysts, especially in case of difficult substrates like aryl amines in which the reaction does not proceed easily with conventional palladium(II) catalysts. In addition to being inexpensive, nickel catalysts can also be removed much easily from the reaction system.

On that basis, Ni(II) preferred catalysts may include, but are not limited to, [1,3-Bis(diphenylphosphino)propane]nickel(II) chloride, Bis(triphenylphosphine)nickel(II) chloride, [1,2-Bis(diphenylphosphino)ethane]nickel(II) chloride, [1,1′-Bis(diphenylphosphino)ferrocene]nickel(II) chloride, and Bis(tricyclohexylphosphine)nickel(II) chloride.

Similarly, Platinum(II) catalysts may be used in this invention, under equivalent reaction conditions. Preferred Pt(II) catalysts include, but are not limited to, Platinum(II) chloride, Platinum(II) bromide, Platinum(II) acetate, Platinum(II) acetylacetonate, Potassium bis(oxalato)platinum(II), Dichloro(cycloocta-1,5-diene)platinum(II), and cis or trans Bis(triphenylphosphine)platinum(II) chloride.

As with palladium(II) catalysts, carbene complexes of platinum(II) prepared by heating platinum bromide with imidazolium salt NHC precursors, and sodium acetate in dimethyl sulfoxide may be used in this invention. Such NHC ligands may be prepared from imidazolium and imidazolinium salts including, but not limited to, 1,3-Bis(2,4,6-trimethylphenyl)imidazolium chloride, 1,3-Bis(2,6-diisopropylphenyl)imidazolium chloride, 1,3-Bis(adamant-1-yl)imidazolium chloride, 1,3-Bis(2,4,6-trimethylphenyl)-4,5-dihydroimidazolium tetrafluoroborate, 1,3-Bis(2,6-diisopropylphenyl)imidazolidinium tetrafluoroborate, 1,3-Bis(2,4,6-trimethylphenyl)imidazolidinium chloride, and 1,3-Bis(2,6-diisopropylphenyl) imidazolidinium chloride.

Amount of catalyst used in this invention may range from 0.05% to 2% per mole of starting amine, and preferably in the range from 0.1% to 1% per mole of starting amine.

An amine and/or a diamine is included in the reaction mixture in stoichiometric amounts (mole per mole) to the quinone.

It is well recognized that, unlike MDI, aliphatic diisocyanates and TDI produced by this invention are low viscosity liquids with relatively high vapor pressures. Therefore, they require special procedures to address potential safety and health hazards associated with their handling and their use.

Processes of the present invention optionally include a blocking agent for blocking isocyanate groups, both aliphatic and aromatic isocyanate groups, providing a novel means to “trap” isocyanate molecules in situ, as they are produced, before they can react with any excess amine, and be subsequently converted into ureas. The resulting blocked isocyanate compound is inert to air, moisture, active hydrogen compounds such as alcohols and amines, and non-hazardous at normal ambient temperatures.

One or more blocking agents is included in amounts up to 10% excess per NCO group (monoamine: 1.1 mole blocking agent per mole of amine-diamine: 2.2 moles blocking agent per mole of diamine).

When heated, with or without catalysts, blocked isocyanates revert to the starting isocyanates and the blocking agents. Free isocyanates can then undergo typical polymerization reactions, such as reactions with polyols or amines to yield polyurethanes or polyureas, respectively.

Blocking agents that may be used in methods according to aspects of the present invention are selected from oximes, phenols, caprolactams, imidazoles and active methylene compounds that generally contain an active hydrogen, said hydrogen being attached to oxygen, sulfur or nitrogen. Blocking agents will react with isocyanate moieties produced in the invention: the product obtained is reversible, that is, it thermally unblocks.

Suitable oximes include, but are not limited to, methylethylketone oxime, acetone oxime, acetaldoxime, formaldoxime and cyclohexanone oxime.

Representative examples of lactams include, but are not limited to, ε-caprolactam, butyrolactam, valerolactam and pyrrolidone. Other lactams which may be used as a blocking agent are as those described in U.S. Pat. No. 4,150,211. ε-caprolactam is a preferred blocking agent because of its ready availability and low cost.

Examples of phenol derivatives include, but are not limited to, phenol, cresol, ethylphenol, butylphenol, nonylphenol, dinonylphenol, styrenated phenol, and hydroxybenzoic acid esters.

Representative blocking agents containing active methylene derivatives which may be used, include, but are not limited to, dimethyl malonate, diethyl malonate, methyl acetoaetate, ethyl acetoacetate, and acetylacetone.

Other examples of blocking agents containing active methylene include, but are not limited to, imidazole, 2-methylimidazole, 2,5-dimethylimidazole, and 3,5 dimethylpyrazole (DMP).

The blocking agent, as described above, is added at a slight stoichiometric excess, preferably from 5 to 10%, over the starting amount of amine.

The isocyanate blocking reaction included according to aspects of methods of the present invention is facilitated by the addition of stannous or diorganotin compounds under catalytic conditions. The more stable diorganotin compounds are preferred in this invention because of their oxidative stability. The compounds that may be used include, but are not limited, to liquid forms of dialkyltins with various fatty acid or mercaptide ligands, such as dimethyltin dilaurate, dioctyltin dilaurate, dibutyltin diacetate, dibutyltin bis(lauryl mercaptide) and dibutyltin bis(isooctylmercaptoacetate). Most preferred compound according to aspects of methods of the present invention is dibutyltin dilaurate (DBTDL).

Addition of amine is preferably performed by introducing the amine, which has been previously diluted in the selected solvent, to the reaction mixture comprising said solvent, catalyst, oxidant, blocking agent, and dissolved carbon monoxide, as described in the following examples.

In order to obtain high isocyanate yield and efficiency, reaction temperatures may be adjusted in the range from 0° C. to 150° C., preferably from 25° C. to 120° C., depending upon reactivity of starting amine, type of solvent, blocking agent, catalyst composition, structure and reactivity.

Embodiments of inventive compositions and methods are illustrated in the following examples. These examples are provided for illustrative purposes and are not considered limitations on the scope of inventive compositions and methods.

### EXAMPLES

### Example 1

In a dry 200-ml, round-bottom, three-necked flask equipped with a magnetic stirring bar, thermometer, an addition funnel a condenser, and a fitted tube connected to a pressure regulator mounted onto a cylinder of carbon monoxide, are placed 0.022 g palladium acetate (0.1 mmole) in 25 ml of acetonitrile and 1.1 g benzoquinone (10 mmole). The reaction mixture is heated to 50° C. until homogeneous; carbon monoxide is then bubbled in at atmospheric pressure. 0.93 g of aniline (10 mmole) dissolved in 25 ml of acetonitrile are placed in the addition funnel and added slowly dropwise to the reaction mixture over a period of approximately 15-30 minutes; carbon monoxide bubbling is continued until the end of the reaction. Progress of the reaction, as measured by the remaining aniline concentration in the flask, is monitored by NMR against an internal standard. Once analysis has shown that there is no remaining aniline, carbon monoxide addition is discontinued and the reaction mixture cooled down to room temperature. Analysis shows production of 0.95 g (8 mmole) of phenyl isocyanate (80% yield, based on aniline added).

### Example 2

In a dry 200-ml, round-bottom, three-necked flask equipped with a magnetic stirring bar, a thermometer, an addition funnel, a condenser, and a fritted tube connected to a pressure regulator mounted onto a cylinder of carbon monoxide, are placed 0.022 g palladium acetate (0.1 mmole) dissolved in 25 ml of acetonitrile, 2.49 g ε-caprolactam (22 mmole-10% excess) and 1.1 g benzoquinone (10 mmole). Carbon monoxide is bubbled in at atmospheric pressure; then the reaction mixture is heated to 80-100° C. 1.22 g of 2,4-diaminotoluene (10 mmole) dissolved in 25 ml of acetonitrile are placed in the addition funnel and added slowly dropwise to the reaction mixture over a period of approximately 15-30 minutes. Carbon monoxide bubbling was continued until the end of the reaction. During the reaction, the isocyanate (—NCO) peak was monitored in the 2250-2270 cm⁻¹ region using FTIR to observe the progress of the blocking reaction which was carried out until disappearance of the —NCO peak.

The white precipitate of blocked aromatic diisocyanate that formed at the end of the reaction was purified by washing with acetone and water for 5 times. Preparation of blocked MDI and 4,4′-dicyclohexyl-methane diisocyanate followed the same reaction conditions.

The blocked aliphatic diisocyanate was washed several times with water for purification.

The obtained powder products were dried in a vacuum oven for 24 hrs at 60° C. Blocked TDI's melting point was 168° C., blocked 4,4′-dicyclohexyl-methane diisocyanate's was 208° C., whereas blocked MDI's melting point was 180° C.

### Example 3

In a dry 200-ml, round-bottom, three-necked flask equipped with a magnetic stirring bar, thermometer, an addition funnel a condenser, and a fitted tube connected to a pressure regulator mounted onto a cylinder of carbon monoxide, are placed 0.054 g [1,3-Bis(diphenylphosphino)propane]nickel(II) chloride (0.1 mmole) in 25 ml of acetonitrile and 1.1 g benzoquinone (10 mmole). The reaction mixture is heated to 50° C. until homogeneous; carbon monoxide is then bubbled in at atmospheric pressure. 0.93 g of aniline (10 mmole) dissolved in 25 ml of acetonitrile are placed in the addition funnel and added slowly dropwise to the reaction mixture over a period of approximately 15-60 minutes; carbon monoxide bubbling is continued until the end of the reaction. Once analysis shows that there is no remaining aniline, carbon monoxide addition is discontinued and the reaction mixture cooled down to room temperature.

### Example 4

In a dry 200-ml, round-bottom, three-necked flask equipped with a magnetic stirring bar, a thermometer, an addition funnel, a condenser, and a fritted tube connected to a pressure regulator mounted onto a cylinder of carbon monoxide, are placed 0.022 g platinum chloride (0.1 mmole) dissolved in 25 ml of acetonitrile, 2.49 g ε-caprolactam (22 mmole-10% excess), 1.1 g benzoquinone (10 mmole) and 0.1 g of DBTDL. Carbon monoxide is bubbled in at atmospheric pressure; then the reaction mixture is heated to 80-100° C. 1.16 g of 1,6-hexamethylene diamine (10 mmole) dissolved in 25 ml of acetonitrile are placed in the addition funnel and added slowly dropwise to the reaction mixture over a period of approximately 15-60 minutes. Carbon monoxide bubbling is continued until the end of the reaction. During the reaction, the isocyanate (—NCO) peak is monitored in the 2250-2270 cm⁻¹ IR region to observe progress of the blocking reaction, which is carried out until disappearance of the —NCO absorption peak. The white precipitate of blocked aromatic diisocyanate that forms at the end of the reaction is purified by washing 5 times with acetone and water.

Any patents or publications mentioned in this specification are incorporated herein by reference to the same extent as if each individual publication is specifically and individually indicated to be incorporated by reference.

The methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

### REFERENCES

(1). (a) Bontempi, A. et al., J. Mol. Catal. 1987, 42, 67. (b) Crotti, C. et al., J. Chem. Soc., Chem. Commun. 1986, 784.
(2). (a) Fukuoka, S. et al., J. Org. Chem. 1984, 49, 1460. (b) Alper, H.; Hartstock, F. W. J. Chem. Soc., Chem. Commun. 1985, 1141. (c) Kanagasabapathy, S. et al., Catal. Lett. 1994, 25, 361. (d) Wan, B. et al., Appl. Catal. A. 1999, 183, 81. (e) Lee, J. S. et al., J. Mol. Catal. 1990, 61, L15. (f) Mulla, S. A. R. et al., J. Mol. Catal. A. 1997, 122, 103. (g) Kim, H. S. et al., Angew. Chem. Int. Ed. 2002, 41, 4300.
(3). Gupte, S. P.; Chaudhari, R. V. Ind. Eng. Chem. Res. 1992, 31, 2069.
(4). Prasad, K. V.; Chaudhari, R. V. J. Catal. 1994, 145, 204.
(5). Jenner, G.; Bitsi, G. Appl. Catal. 1987, 32, 293.
(6). Shi, F.; Deng, Y. J. Catal. 2002, 211, 548.
(7). Giannoccaro, P. J. Organomett. Chem. 1987, 336, 271.
(8). Chen, B.; Chuang, S. S. C. J. Mol. Catal. A. 2003, 195, 37.
(9). (a) Skupinska, J. et al., Appl. Catal. A. 2005, 284, 147. (b) Skupinska, J.; Karpinska, M. Appl. Catal. A. 2004, 267, 59. (c) Skupinska, J. et al., React. Kinet. Catal. Lett. 2004, 82, 311.
(10). Wan, B. et al., Appl. Catal. A. 1999, 183, 81.
(11) (a) Gabriele, B G. et al., Topics in Organometallic Chemistry 2006, 18, 239-272; (b) Gabriele, B. et al., Synlett, 2004, 2468-2483; (c) Gabriele, B. et al., Current Organic Chemistry, 2004, 8, 919-946.
(12) (a) Orito, K. et al., Org. Chem. 2006, 71(16), 5951-5958; (b) Zheng, S. et al., Helv. Chim. Ac, 2007, 90, 1471-1476; (c) Zheng, S.; Li, F.; Liu, J.; Xia, C. Tetrahedron Lett., 2007, 48, 5883-5886. (d) Gabriele, B. et al., Chem. Commun., 2003, 486-487; (e) Gabriele, B. et al., Org. Chem. 2004, 69(14), 4741-4750. (f) McCusker, J. E. et al., J. Org. Chem. 2000, 65, 5216-5222; (g) McCusker, J. E.; Qian, F.; McElwee-White, L. J. Mol. Cat. A. Chemical 2000, 159, 11-17; (h) Zhang, Y. et al., Green Chem., 2005, 7, 451-455. (i) Mizuno, T.; Mihara, M.; Iwai, T.; Ito, T.; Ishino, Y. Synthesis, 2006, 17, 2825-2830; (j) Mizuno, T. et al., Heteroatom Chemistry, 2009, 20(1), 64-68.
(13) Mizuno, T. et al., Synthetic Communications 2000 30(9), 1675-1688.
(14) Katritzky, A. R. et al., Bioorg. Med. Chem. Lett. 2002, 12, 3453-3457.
(15) Gabriele, B. et al., Org. Chem. 2004, 69(14), 4741-4750.
(16) U.S. Pat. No. 6,781,010 B1, 8/2004, Mason
(17) Vetter, J.; A safer route to MDI—An assessment of a phosgene free manufacturing process; Rijksuniversiteit, Groningen, The Netherlands, 2010
(18) H. van den Berg et al., Phosgene free route to Methyl Diphenyl Diisocyanate (MDI): A technical and economical evaluation, Chemical Engineering Journal, Volume 207-208, issue (Oct. 1, 2012), p. 254-257.
(19). Chiarottoo, I.; Feroci, Marta. J. Org. Chem. 2003, 68, 7137-7139.
(20) Z. Wicks, Prog. Org. Coat., 3, 73 (1975).
(21) Z. Wicks, Prog. Org. Coat., 9, 3 (1981).
(22) D. A. Wicks and Z. W. Wicks, Jr., Prog. Org. Coat., 36, 148 (1999).
(23) D. A. Wicks and Z. W. Wicks, Jr., Prog. Org. Coat., 41, 1 (1999).

1. A method for production of an isocyanate, comprising:
reacting a homogeneous reaction mixture comprising a monomeric or oligomeric primary amine or diamine, carbon monoxide, a Group X divalent noble metal compound or complex catalyst, a quinone oxidizing agent and a polar aprotic solvent, thereby producing an isocyanate.

2. The method of claim 1, wherein the reaction mixture is substantially phosgene free.

3. The method of claim 1, wherein the carbon monoxide is at atmospheric pressure.

4. The method of claim 1, wherein the Group X divalent noble metal compound or complex catalyst comprises nickel(II), platinum(II) and/or palladium(II).

5. The method of claim 1, further comprising reacting the isocyanate with a blocking agent, producing a blocked isocyanate.

6. The method of claim 1, wherein the catalyst is present in an amount in the range of 0.05% to 2% per mole of the monomeric or oligomeric primary amine or diamine.

7. The method of claim 1, wherein the reacting is under reaction conditions comprising a temperature in the range of 0° C.-150° C.

8. The method of claim 1, wherein the quinone oxidizing agent is present in a stoichiometric amount per mole of the monomeric or oligomeric primary amine or diamine.

9. The method of claim 1, wherein the quinone oxidizing agent is present in an amount in the range of 0.05% to 2% per mole of the monomeric or oligomeric primary amine or diamine, and wherein a stoichiometric amount of manganese oxide is present relative to the amount of the monomeric or oligomeric primary amine or diamine.

10. A method for production of an isocyanate, comprising:
reacting a homogeneous reaction mixture comprising a monomeric or oligomeric primary amine or diamine, carbon monoxide, a Group X divalent noble metal compound or complex catalyst, an oxidizing agent and a polar aprotic solvent, thereby producing an isocyanate, wherein the reaction mixture is substantially CuCl₂ free and substantially HCl free.

11. The method of claim 10, wherein the reaction mixture is substantially phosgene free.

12. The method of claim 10, wherein the carbon monoxide is at atmospheric pressure.

13. The method of claim 10, wherein the Group X divalent noble metal compound or complex catalyst comprises nickel(II), platinum(II) and/or palladium(II).

14. The method of claim 10, further comprising reacting the isocyanate with a blocking agent, producing a blocked isocyanate.

15. The method of claim 10, wherein the catalyst is present in an amount in the range of 0.05% to 2% per mole of the monomeric or oligomeric primary amine or diamine.

16. The method of claim 10, wherein the reacting is under reaction conditions comprising a temperature in the range of 0° C.-150° C.

17. The method of claim 10, wherein the reacting is under reaction conditions comprising a temperature in the range of 25° C.-120° C.

18. The method of claim 10, wherein the oxidizing agent is a quinone.

19. The method of claim 18, wherein the quinone oxidizing agent is present in a stoichiometric amount per mole of the monomeric or oligomeric primary amine or diamine.

20. The method of claim 18, wherein the oxidizing agent is present in an amount in the range of 0.05% to 2% per mole of the monomeric or oligomeric primary amine or diamine, and wherein a stoichiometric amount of manganese oxide is present relative to the amount of monomeric or oligomeric primary amine or diamine.
